(19) United States
(12) Patent Application Publication
(10) Pub. No.: US 2012/0207850 A1
(43) Pub. Date: Aug. 16, 2012

(54) Methods for Increasing the Kinetic Activity of Alcohol, Water and Other Liquids, so as to Render the Liquids More Useful in Enhancing the Alternative Cellular Energy Pathway in the Prevention and Therapy of Diseases

(76) Inventor: William John Martin, South Pasadena, CA (US)

(21) Appl. No.: 13/029,116

(22) Filed: Feb. 16, 2011

(51) Int. Cl. A61K 33/00; A61Q 19/00; A61K 8/34; A61K 31/045; A61K 8/19; A61P 17/02; A61P 3/04

(52) U.S. Cl. 424/600; 514/724

(57) ABSTRACT

The kinetic energy of liquids can be increased by several methods described in the present application. The methods include exposure to a magnetic field provided by a rotating or vibrating magnet; exposure to an electromagnetic field provided by an electrical power cord; and placement of the liquid within the vicinity of electrostatic energy and/or sound energy. A previously described method is the bubbling of electrolysis-generated water gas (Brown's Gas) into the liquid. The increased kinetic energy can be demonstrated using a previously described neutral red dye kinetic assay (NR-Kinetic assay). Energized liquids include alcohol, alcoholic beverages and water. The use of the energized solutions to enhance the alternative cellular energy (ACE) pathway in the therapy of individuals is described.

### CROSS REFERENCE TO RELATED APPLICATIONS

Co-Pending Patent Application

Methods for Detection of Ultraviolet Light Reactive Alternative Cellular Energy Pigments (ACE-pigments) William John Martin Submitted Dec. 24, 2007. Publication number 20090163831

Method of assessing and of activating the alternative cellular energy (ACE) pathway in the Therapy of Diseases. William John Martin Submitted Jan. 16, 2008. Publication number 20090181467

Enerceutical mediated activation of the alternative cellular energy (ACE) pathway in the therapy of diseases. Submitted May 8, 2008. Publication number 20090280193

Enerceutical activation of the alternative cellular energy (ACE) pathway in therapy of diseases. Submitted Feb. 11, 2009. Publication number 20090202442

Method of using the body's alternative cellular energy pigments (ACE-pigments) in the therapy of diseases Submitted Feb. 20, 2009. Publication number 20100215763

Urine as a source of alternative cellular energy pigments (ACE-pigments) in the assessment and therapy of diseases. Submitted Mar. 5, 2009. Publication number 20100196297

Diagnostic value of systemic ACE pathway activation in the detection by fluorescence of localized pathological lesions. Submitted Jul. 26, 2010. Publication number 20100291000

Enerceutical mediated activation of the alternative cellular energy (ACE) pathway in the therapy of diseases. Submitted July 2010.

Energy Charged Liquids to Enhance Enerceutical Activation of the Alternative Cellular Energy (ACE) Pathway in the Therapy of Diseases. Submitted Dec. 17, 2010. Application Ser. No. 12/972,344

Energy Charged Alcoholic Beverages for Enhancing the Alternative Cellular Energy Pathway in the Prevention and Therapy of Diseases. Submitted January 2011. Application number

Methods for Detecting and Monitoring the Activity of Energized Water and Other Liquids Useful for Enhancing the Alternative Cellular Energy Pathway in the Prevention and Therapy of Diseases. Submitted February 2011

### STATEMENT REGARDING FEDERALLY SPONSORED RESEARCH OR DEVELOPMENT

Not applicable: No Federal funding was received in support of this patent application.

### REFERENCE TO SEQUENCE LISTING, A TABLE OR A COMPUTER PROGRAM LISTING COMPACT DISK APPENDIX

Not applicable.

### BACKGROUND OF THE INVENTION

In recently submitted co-pending patent applications, which are incorporated by reference herein, I disclosed that the passage of electrolytically derived Water Gas (otherwise known as Brown's Gas) into alcohol (200 proof absolute ethanol) greatly enhances the ability of the alcohol to react with neutral red dye and with alternative cellular energy (ACE) pigments (defined in the co-pending patent applications). When a small number (usually around 10) of neutral red dye particles (obtained directly from a commercially available neutral red dye powder e.g. Dudley Corp. N.J.), are gently sprinkled onto a plastic dish, which contains Water Gas “charged” alcohol; and the solution is illuminated with visible and/or UV light, there is rapid and vigorous dissolving of most of the material within the neutral red dyes particles. This occurs in a directional manner forming long, narrow red streams of dissolved neutral red. Equally impressive, fine particles, breaking away from the larger particles of neutral red dye, which remain non-dissolved in the alcohol solution, undergo continuing to-and-fro movements, with apparent attractive forces rapidly alternating with repulsive forces. The linearity of the dissolving neutral red and the dynamic movements of non-dissolved particles occur in regular (non-charged) absolute alcohol, but are greatly heightened by Water Gas charging of the alcohol. The use of charged alcohol also significantly enhances the intensity of the orange fluorescence of the neutral red solution, when compared with dye dissolved in non-charged alcohol. As the alcohol (both charged and non-charged) finally evaporates, the precipitating neutral red dye assumes attractive, banded circular patterns, as if being influenced by an interactive energy field. The patterns are more striking using charged alcohol. The charged alcohol also shows greater interaction than does non-charged alcohol with ACE pigments collected from the saliva and/or perspiration of virus infected patients. When used therapeutically, the combination of neutral red with charged alcohol is decidedly more effective in enhancing the ACE pathway in energy deficient individuals, when compared with, still quite impressive, benefits obtained using untreated (non-charged) alcohol.

The linear dissolving pattern of neutral red dye observed in alcohol solutions, was also observed in various alcoholic beverages, especially when they were charged with Water Gas. This extraordinary pattern is not ordinarily seen when neutral red dye is added to ordinary (non-charged) water in similar plastic dishes. Instead, slowly dissolving neutral red particles become gradually surrounded by the dissolved dye in essentially concentric discs of red dye. An exception is seen with some plastic drinking glasses and some plastic covered paper cups. In these containers, there is an obvious attraction between the plastic material and the neutral red particles, leading to radial movements of the particles towards the interface between the surface of the water and the plastic container. (These types of containers are, therefore, unsuitable for used in the experiments described in this application). I have commonly used small (1.5″) square individual polycarbonate dishes and various multi-well tissue culture dishes. The dishes are generally observed using a low power, dissecting microscope with spacing between the light and the dish to help reduce heat transfer. In some experiments light emitting diode (LED) illumination was used to exclude heat, as opposed to light, as the cause of particle movements.

An important finding was observed when neutral red dye particles, are gently sprinkled (scattered) onto a plastic dish of distilled water, and the dish is placed in close proximity to a dish of charged alcohol with moving neutral red dye particles. Instead of the particles in the water remaining essentially stationary and only slowly dissolving, several of the particles will begin to move throughout the water. Moreover, if additional neutral red dye is added, the fresh particles will show more linear dissolving patterns with movements of the remaining non-dissolved particles. The energy transference effect is best seen when the dishes are in direct contact or comprise adjacent wells within the same multi-well dish. Physical contact is not absolutely necessary, however, and the effect has been seen between separated dishes. I, thereby, discovered that the altered dissolving pattern of neutral red dye in ordinary water potentially provided a simple assay for detecting a radiating energy coming from the well containing the charged alcohol and neutral red particles. The assay also provides a means of detecting the water and alcohol energizing capacities of various additional forms of energy, in addition to that provided by Water Gas. For convenience, this assay will be subsequently referred to as the “NR-Kinetic Assay.”

Specifically, the present patent application explores various alternative methods to the use of Water Gas to enhance the kinetic activity of water, alcohol and alcoholic beverages. Effective methods included the use of magnetic, electromagnetic, electrostatic and sound energies. Moreover, application of these methods was shown to enhance the capacity of alcohol plus dissolved neutral red dye to activate the ACE pathway in an autistic patient, with an impaired ACE pathway. Ongoing clinical studies are also being performed on variously energized water samples.

The research has helped in understanding many of the previously reported health enhancing and plant growth promoting activities of water. Some of the claims simply relate to where the water is collected, implying possible geological influences on the water. Numerous methods have been stated to convert ordinary water into a more “energized” form. These methods include electrolysis with partitioning of drinkable alkalize water from non-drinkable acidic water; the use of a vortex to spin water; magnets, supposedly useful to align water molecules; electromagnetic energies, including ultraviolet, visible light, infrared and radio wave transmissions, supposedly to impart specific resonance to the water; and the addition of various components. Among the added components to potentially “activate” water are minerals, particularly magnesium and chlorides; humic and fulvic acids; zeolites; gases, including hydrogen and Water Gas; and undisclosed “proprietary” materials. Short of performing rigorous clinical trials, health claims of the benefits of the various water processing methods, are difficult to substantiate.

There is also an abundance of speculations as to how energized water may differ from ordinary water. These include a possible lower density from actual expansion of the water molecules into a more linear form. It has also been suggested that there may be actual dissociation of one of the hydrogen atoms from oxygen and its combining with the other hydrogen in the water molecule to create a HHO, rather than an HOH linkage. The formation and sizes of water clusters, either as clathrates (cages) or dendrimers, may also be changed by the absorption of energy. Measurements such as pH, Redox (ORP), surface tension, conductivity, supposed cluster size as assessed by nuclear magnetic resonance (NMR), etc., have not been shown to reliably differentiate between supposedly modified and health-enhancing beneficial water from ordinary unprocessed water.

There is, therefore, a need for a more meaningful assay of water activity, which can reasonably relate to its health enhancing and plant growth promoting activities. The NR-Kinetic Assay has been disclosed as a simple screening method to assess both naturally occurring and processed water samples for their potential to provide therapeutic benefits, presumably through the ACE pathway.

The present patent application disclosed the use the NR-Kinetic assay system to investigate the ability of magnetic, electromagnetic, electrostatic and sound energies to impart energy not only into water but also into alcohol and alcoholic beverages. Moreover, the energized alcohol has shown superior therapeutic properties, when compared with non-energized alcohol, in a neutral red dye/UV light procedure, when applied to a child with autism.

### BRIEF SUMMARY OF THE INVENTION

The NR-Kinetic assay was used to demonstrate easily discernable increased activity in variously treated versus untreated control liquids. In an initial series of experiments, alcohol (200 proof ethanol), various alcoholic beverages and water were energized using a rotating and/or rapidly vibrating magnet. The treated solutions displayed markedly enhanced activity in the NR-Kinetic assay, when compared to untreated solutions. Solution activation was also achievable using placed next to sound frequencies generated from computer speakers. Even electromagnetic energy provided by a electrical power cord also provided some evidence of both water and alcohol, NR-Kinetic activation. Finally, an electrostatic field generated by a van de Graaff generated was successfully tested. Importantly, the magnetic, sound and electromagnetic field energized alcohols were found to be superior to unprocessed alcohol when neutral red dye was added, and the solutions poured into Ziploc bags, which were placed onto the soles of the feet of an autistic child and illuminated with ultraviolet (UV) light.

### BRIEF DESCRIPTION OF THE DRAWINGS

Not Applicable and none included

### DETAILED DESCRIPTION OF THE INVENTION

The NR-Kinetic Assay has previously been employed in the direct assessment of various kinds of processed liquids, including absolute alcohol and alcoholic beverages. For instance, it was also used to demonstrate the marked effect of simply bubbling Water Gas through absolute alcohol on the ability of the “charged” alcohol to react with neutral red dye. Not only was there more of a direct effect on neutral red added to the Water Gas charged alcohol, such as more intense fluorescence, but the light illuminated charged alcohol solution containing neutral red dye, was clearly able to distantly induce movements of non-dissolved neutral red dye particles placed in an adjacent well containing regular water. In this assay, the light illuminated neutral red dye containing charged alcohol was placed in one of the wells of a tissue culture plate. Adjacent wells contained water plus stationary un-dissolved neutral red dye particles. These wells were observed microscopically to see whether any of the non-dissolved neutral red particles would begin to move, which they did beginning approximately 30-60 seconds later. An additional observation on the adjacent neutral red dye in water wells included the occasional formation of numerous gas bubbles, which might well represent the formation of Water Gas. The energy transference type of assay consistently provided positive results, which were not seen using multiple wells in other culture dishes, which simply had water plus neutral red solutions. The Water Gas charged alcohol was consistently judged to be superior to unprocessed alcohol when tested on a child with autism, using a standardized protocol, described in prior patent applications.

The availability of the NR-Kinetic assay has allowed for the testing of various other suggested methods for energizing water and for pioneering these methods for the purpose of activating alcohol, alcoholic beverages and potentially all other types of liquids. I began these studies using a rotating bar magnet placed into a small container of either water or alcohol. The container was placed onto a magnetic stirring plate (Haakebuchler Instruments Inc. MR 2002) and the magnet rotated at speeds up to 1,000 turns per minute. To control for the vortex effects of the magnetic stirring, a small bar magnet, coated on one side with porcelain, was more commonly used. It did not rotate, but rapidly vibrated. Interestingly, when in water but not alcohol, and only when magnetically vibrated, very small bubbles would form on the sides and top of the magnet, occasionally dislodging and rising to the surface. Although various times were tried, including overnight, an effect on the water was readily seen at 30 minutes. To show an effect, a sample of the treated water or alcohol was placed into a plastic dish or into one of the wells of a multi-well tissue culture dish, e.g. Costar 24, 48 or 96 well plates or Falcon 6 or 24 well plates. A small number of neutral red particles were added and their movements observed microscopically using an inverted microscope Leica, with adjustable magnification. The multi-well plates were used to show a nearby effect on neutral red particles placed into ordinary water in an adjacent well as compared with neutral red particles placed into water in a more distant well, or in a separate multi-well plate. Again, there was an interesting observation that for reasons not immediately apparent, sometimes-significant background NR-Kinetic activity was discernable in ordinary tap water, which had not been purposefully charged. At other times, non-processed tap water would show no activity. A jug containing filtered water provided a more consistent negative control for most of the experiments involving water. For studies using alcohol (200 proof ethanol), there was always quite marked background NR-Kinetic activity. This activity was markedly enhanced when the alcohol was subject to a vibrating magnet, although still not as marked as alcohol, through which Water Gas had been bubbled. Magnetic activation has persisted for several days, allowing for clinical testing. The third criterion of activation of water and especially of alcohol was the banding pattern shown by the neutral red dye as it precipitated from solutions allowed to evaporate to dryness. In some experiments, alcoholic beverages (whisky, gin, vodka and rum) were used instead of absolute alcohol, with consistent, supportive findings of magnetic activation, as assessed by using the NR-Kinetic assay.

In the next series of experiments, a computer sound generator software program (Marchand Function Generator) was used to generate sounds at various controllable frequencies. Dishes containing water, alcohol or alcoholic beverages, plus a few particles of neutral red dye, were observed microscopically to assess the dissolving pattern (whether or not long linear streaks were being formed and the to-and-fro movements of non-dissolved neutral red dye particles). The computer sound system (Marchand) was activated and the speakers placed close to the dish being microscopically observed. Using a computer mouse control, the sound frequencies being applied was varied throughout the range of 1-20,000 Hz . Unlike similar experiments using actual ACE pigment particles, no actual resonance frequency was observable for the particles of neutral red dye. However, it did appear that over certain ranges of frequencies, the movements of the particles were more energetic than at other frequencies. After several experiments, a decision was made to use saw tooth sound at 500 Hz to produce liquids for more detained testing. Ordinary tap water and absolute alcohol were exposed to 30 minutes of sound at 500 Hz. With both liquids, there was a clearly discernable enhanced NR-Kinetic activity, when compared to control fluids maintained well away from the computer-generated sound.

The final two approaches at energizing water and alcohol solutions comprised testing for the influence of electromagnetic and electrostatic energy fields on the performance of the liquids in a subsequent NR-Kinetic assay. For the electromagnetic field, I simply placed a small container of the solution onto an electrical power cord while it was being used to run a major appliance, e.g., desk top computer, TV, washer/dryer, etc. I did so for approximately 30 minutes prior to testing with the NR-Kinetic assay. Separately placed containers of the same solution (either water or alcohol or in some experiment, an alcoholic beverage) provided suitable controls. The electromagnetic field from the electrical power cords definitely enhanced the linear streaking and particle motility of the dissolving neutral red dye. For an electrostatic field, I used a hobby van de Graaff generator (15″ high with a 4″ metal dome collector and powered by 4 Type C batteries). The containers were tapped to the top of the dome of the generator, which was run for 15 minutes, and removed after an additional 15 minutes. This procedure also enhanced the performance of the liquids in the NR-Kinetic assay, but was considered impractical for routine use.

The method of magnetic activation of water and alcohol compared favorably with the previously described method of bubbling electrolytically generated Water Gas into the solutions, at least in terms of the NR-Kinetic assay. The method of sound activation was less easily defined in terms of an actual optimal frequency. The placement of a solution next to a electrical power cord was the easiest to perform, but less effective than the other methods. Altogether, I concluded that each of the methods essentially involved a conversion of an input energy into a kinetic energy, which could be stored by the liquid and released under various circumstances, including the addition of neutral red dye with illumination. Moreover, the neutral red dye captured kinetic energy was itself able to transmit kinetic energy to more distantly placed liquids. The primary purpose of the project has been to devise effective means of activating the body's ACE pathway in the therapy of infectious and other diseases, enhancement of wound healing through a non-inflammatory process, etc. The study was, therefore, extended to an assessment of the various methods of energizing alcohol in the neutral red dye/UV light assay, as was being applied to a child with autism.

For this study, samples of alcohol (200 proof ethanol) were activated over 30 minute periods using i) vibrating bar magnet (M); ii) 500 Hz sound (S); iii) placement onto an electrical power cord (E); or iv) bubbling of Water Gas generated from citric acid:potassium carbonate containing water (G). The mother of a 15-year-old autistic child subsequently tested the coded samples. The mother had been using the neutral red dye/UV light therapy protocol for over 2 years and is extremely reliable in her reporting. The four samples were sequentially tested in the same day for the purpose of comparisons. Overall, the mother reported never-before-seen quite remarkable improvements, with the indication that the magnetic activated alcohol was comparable, if not superior to the Water Gas treated alcohol. She also reported more noticeable effects with the sound energy and electromagnetic field exposed samples, than she had previously noted with unprocessed alcohol. Literally, for the first time she heard her daughter singing in tone. She also noted improved facial appearance and mannerisms. She further commented upon the exceptionally good emotional connection, steady eye contact, and excellent pronunciation of complex words and, remarkably, singing with far greater tonal range than the mother had ever previously heard from her child. All of the samples provided marked benefit to the autistic child when used in a Ziploc bag with neutral red dye, tapped onto the soles of her feet and illuminated with a UV light. Of the various samples, the magnetic activation was judged to be the best, an opinion shared by the child.

The methods energizing water using Water Gas, magnetic, electromagnetic and sound energies are also being applied to drinking water, including some water that is being produced in a water bottling plant. The NR-Kinetic assay is being used to help discern the relative benefits of various processes. Adjustments are being made to make the methods more compatible with the existing and recently installed equipment. For example, the vortexing of water in a fixed magnetic field can be considered the equivalent of a moving magnet in stationary water. While it is likely that Water Gas, magnetism, sound and various forms of electromagnetic energies are all affecting water through a final common mechanism, some of the procedures may still be additive and not necessarily duplicative. Clinical studies using energized water, using the methods described in this patent application, and which have strong demonstrated activity in the NR-Kinetic Assay, have been approved and will be underway shortly. Included in these studies is the use of water for consumption, as well as a potential cosmetic. Recipients will also be monitored for neutral red dye induced fluorescence of saliva samples, as per earlier patent applications.

The NR-Kinetic assay is also contributing to a better understanding of alcohols and of other organic liquids (acetone, acetic acid, etc.). I have shown that these liquids can also capture, store and transmit physical energies, similar to those, which can energize water. It was recently reported that in presence of certain metals, alcohol has the capacity to catalyze the formation of carbon to carbon bonding. Quite possibly alcohol can do so more readily if energized, using Water Gas, magnets, sound or electromagnetic energies. Of immediate application is the use of energized alcohol in therapeutic efforts to activate the ACE pathway in individuals with various illnesses or otherwise impaired optimal functioning. Clinical studies are also being planned comparing the benefits of consuming Water Gas activated water with magnetic and sound activated water. The NR-Kinetic assay is currently being used to assess water from a water bottling plant using a process, which combines vortexing of the water within a column of magnetic material, exposure of the water to other magnetic devices, addition of fulvic acid and infusion of Water Gas. Once optimized, the water will be evaluated for its ACE enhancing benefits in controlled clinical trials.

The principles, preferred embodiments and modes of operation of the present invention, intended to be protected herein, is not to be construed as limited to the use of magnetic and sound energies to enhance the energetic activity of liquids, including water and alcohol, intended for the activation of the ACE pathway The patent application is not restricted to the use of neutral red as the indicator dye in the kinetic assay, since several other dyes can and have been used in similar experiments. Neutral red has the advantage, however, of potentially being used in therapeutic endeavors aimed at activating the ACE pathway. ACE pigments, collected from a patient's saliva or perspiration, are also anticipated as being more active in energized, compared with regular water and alcohol. The term “energized” is also not meant to be exclusive of other relevant terms, which I or other investigators may use in furthering the research described in this application.

Additional modifications of the basic tenets disclosed in the present patent application will readily occur to those skilled in the art and especially upon practicing the currently described methods. For example, magnets can be in a fixed location and the water or alcohol rotated. Or magnetic fields can be pulsed by alternating electrical fields. Energized water and alcohol are being used separately and in combination as cosmetics and body washes. Wounds are also being treated with these solutions. Therefore, many variations and changes may be made without departing from the scope and spirit of the invention as encompassed by the appended claims.

1. A method of treating an individual, comprising the use of a liquid solution, which has been processed in such a manner that it will show enhance kinetic linear and to-and-fro oscillatory movement of particles of an added dye, such as neutral red dye, and further comprising the use of the solution in a procedure, which results in local and/or systemic activation of the alternative cellular energy (ACE) pathway of the treated individual.

2. The method of claim 1, in which the solution is an alcohol and the processing technology is the exposure of the alcohol to a vibrating and/or rotating magnet of sufficient strength or duration so as to enhance the kinetic linear and to-and-fro oscillatory movement of particles of an added dye, such as neutral red dye.

3. The method of claim 1, in which the solution is an alcohol and the processing technology is the exposure of the alcohol to a sound energy of an appropriate frequency and of sufficient strength and duration so as to enhance the kinetic linear and to-and-fro oscillatory movement of particles of an added dye, such as neutral red dye.

4. The method of claim 1, in which the solution is an alcohol and the processing technology is the exposure of the alcohol to an electromagnetic field of an appropriate frequency and of sufficient strength and duration so as to enhance the kinetic linear and to-and-fro oscillatory movement of particles of an added dye, such as neutral red dye.

5. The method of claim 1, in which the solution is an alcohol and the processing technology is the exposure of the alcohol to an electrostatic field of sufficient strength and duration so as to enhance the kinetic linear and to-and-fro oscillatory movement of particles of an added dye, such as neutral red dye.

6. The method of claim 1, in which the solution is water and the processing technology is the exposure of the alcohol to a vibrating and/or rotating magnet of sufficient strength or duration so as to enhance the kinetic linear and to-and-fro oscillatory movement of particles of an added dye, such as neutral red dye.

7. The method of claim 1, in which the solution is water and the processing technology is the exposure of the alcohol to a sound energy of an appropriate frequency and of sufficient strength and duration so as to enhance the kinetic linear and to-and-fro oscillatory movement of particles of an added dye, such as neutral red dye.

8. The method of claim 1, in which the solution is water and the processing technology is the exposure of the alcohol to an electromagnetic field of an appropriate frequency and of sufficient strength and duration so as to enhance the kinetic linear and to-and-fro oscillatory movement of particles of an added dye, such as neutral red dye.

9. The method of claim 1, in which the solution is water and the processing technology is the exposure of the alcohol to an electrostatic field of sufficient strength and duration so as to enhance the kinetic linear and to-and-fro oscillatory movement of particles of an added dye, such as neutral red dye.

10. The method of claim 1, in which the procedure comprises the use of the processed liquid, preferably an alcohol, with the addition of a small quantity (approximately 0.1-1.0 mg/ml) of added neutral red dye, which the solution being either directly applied to an area of the body, or placed within a container which is laid onto the body; followed by ultraviolet light illumination of the solution in such as manner, which results in local and/or systemic activation of the alternative cellular energy (ACE) pathway of the treated individual.

11. The method of claim 1, in which the procedure comprises the use of the processed liquid, preferably water, which is ingested in a sufficient quantity result in the local and/or systemic activation of the alternative cellular energy (ACE) pathway of the treated individual.

12. The method of claim 1, in which the activation of the alternative cellular energy (ACE) pathway of the treated individual is intended as a means of tissue regeneration and/or wound repair.

13. The method of claim 1, in which the activation of the alternative cellular energy (ACE) pathway of the treated individual is intended as a means of enhancing physical appearance through an improvement in complexion.

14. The method of claim 1, in which the activation of the alternative cellular energy (ACE) pathway of the treated individual is intended as a means of reducing an excessive apatite for food, which can be driven by a deficiency of the ACE pathway.
